(19) United States
(12) Patent Application Publication
(10) Pub. No.: US 2016/0310297 A1
(43) Pub. Date: Oct. 27, 2016

(54) UNIVERSAL FORCE SENSOR FOR MEDICAL APPLICATIONS

(71) Applicants: John Anes, Folsom, CA (US); Nicholas Stephen Baker, Reno, NV (US); Scott Decker, El Dorado Hills, CA (US); Rida Hariri, El Dorado Hills, CA (US); Kenneth D. Johannaber, Reno, NV (US); Steven King, El Dorado Hills, CA (US); Andrew P. Sizelove, Reno, NV (US)

(72) Inventors: John Anes, Folsom, CA (US); Nicholas Stephen Baker, Reno, NV (US); Scott Decker, El Dorado Hills, CA (US); Rida Hariri, El Dorado Hills, CA (US); Kenneth D. Johannaber, Reno, NV (US); Steven King, El Dorado Hills, CA (US); Andrew P. Sizelove, Reno, NV (US)

(21) Appl. No.: 15/134,810

(22) Filed: Apr. 21, 2016

Related U.S. Application Data
(60) Provisional application No. 62/150,968, filed on Apr. 22, 2015.

(51) Int. Cl. A61F 2/46; A61F 2/38; G01L 5/00

(52) U.S. Cl. CPC A61F 2/4684; G01L 5/00; A61F 2/4603; A61F 2/38; A61F 2/468; A61F 2002/30672

(57) ABSTRACT

Devices, systems, and methods are provided to delivery force sensing within provisional prosthesis and surgical instruments. A system can include a universal force sensor module and a trial prosthesis. The universal force sensor module can include an upper module cover, a module base, a force sensor, a circuit board, and a battery. The upper module cover can include a cover flex section surrounding a cover instrument contact surface. The module base can include a sensor support structure and be configured to receive a cover perimeter section of the upper module cover. The force sensor can be disposed within the module base and supported by the sensor support structure. The circuit board can be electrically coupled to the force sensor and the battery. The trial prosthesis can include a sensor module cavity adapted to securely retain the universal force sensor module.

### CLAIM OF PRIORITY

This application claims the benefit of U.S. Provisional Patent Application Ser. No. 62/150,968, filed on Apr. 22, 2015, the benefit of priority of which is claimed hereby, and which is incorporated by reference herein in its entirety.

### BACKGROUND

During orthopedic procedures such as knee replacement or similar joint reconstruction surgeries, various tools and instruments are used to assist with the various procedures, including the use of force sensors built into provisional or trial implant prosthetics to assist surgeons in balancing soft tissues. For example, during a conventional total knee arthroplasty (TKA) procedure provisional components can be placed on the distal end of the femur and proximal end of the tibia to confirm proper sizing and fit. Provisional components come in a wide variety of sizes and shapes to match the permanent components of the final implant. In some examples, the provisional or trial components can have force sensing components designed in to further assist the surgeon in determining the proper size, orientation, and configuration of the permanent components. The eLIBRA Dynamic Knee Balancing System from Zimmer of Warsaw, IN is an example of knee trialing instrumentation that includes designed in force or pressure sensing capabilities for assisting in balancing soft tissues in the knee. An example of the eLIBRA Dynamic Knee Balancing system is detailed in U.S. Pat. No. 7,442,196.

### OVERVIEW

Assemblies, configurations, and methods of use for a universal force sensor module usable across a wide range of medical device applications. In an example, the universal force sensor module provides a convenient, compact, and self-contained module that can be interchangeably inserted into a range of devices, such as trial prosthetic devices for various joint reconstruction surgeries. The self-contained sensor module is battery operated and wirelessly communicates with an external computing device to provide force information within the trial prosthetic. An external computing device can perform complex calculations, render graphical interfaces, and store surgical information related to force data provided by the self-contained sensor modules. The sensor modules are designed to enable an external computing device to monitor multiple individual sensor modules concurrently, allowing a trial prosthetic to include as many individual sensor modules as necessary to provide the surgeon with useful soft tissue or joint operation information while in place. Obtaining data from multiple locations within a joint can enable an external computing device to perform additional calculations based on known relationships between the sensor locations within the joint. The universal sensor modules provide a simple single device for integration into a wide range of trial or provisional prosthetics or other instrumentation without the need to design specific trials or provisionals with force sensing integrated into the devices. The use of interchangeable universal sensor modules simplifies inventories and use in the surgical setting.

The present inventors have recognized, among other things, that the currently available force sensing devices require a high degree of initial design for each variation of trial or provisional prosthetic and require instrument kits or systems to contain multiple expensive sensor integrated provisionals to accommodate various provisional types (ex: level of implant constraint) or different sizing, which deters use and adoption of force sensing for joint reconstruction and other similar procedures. These problems, and others, are solved by the present invention through the ability to integrate compact universal force sensing modules into a wide variety of provisional prosthetics, while only requiring a limited number of sensor modules to be included within a surgical kit or system. Separating the force sensing circuitry into self-contained modules limits the need to include expensive circuitry across the entire range of provisional prosthetics and may provide greater control and calibration of the individual force sensing components. Use of individual sensor modules also provides a surgeon options for when and how to use force sensing within any given procedure, if force sensing is not needed the surgeon can simply insert a plug in place of the universal sensor module. Additional benefits arising from the present subject matter will be obvious to one of ordinary skill in the art when reading the remainder of the detailed description.

The following, non-limiting examples, detail certain aspects of the present subject matter to solve the challenges and provide the benefits discussed herein, among others.

Example 1 can include a universal force sensor module. The universal force sensor module can include an upper module cover including a cover flex section. The universal force sensor module also includes a module base including a sensor support structure, the module base configured to receive a cover perimeter section of the upper module cover. The universal force sensor module further includes a force sensor disposed within the module base and supported by the sensor support structure, and a circuit board electrically coupled to the force sensor and a battery, the circuit board and battery disposed within the module base.

In Example 2, the universal force sensor module of example 1 can optionally include a cover flex section with a reduced thickness portion surrounding a cover instrument contact surface.

In Example 3, the universal force sensor module of example 2 can optionally include a reduced thickness portion of the cover flex section that allows for micron-level defections in the cover instrument contact surface in a direction substantially perpendicular to a plane formed by the outer upper surface of the upper module cover.

In Example 4, the universal force sensor module of any one of Examples 1 and 3 can optionally include an upper module cover that further includes a sensor interface structure disposed on the inside surface and configured to contact the force sensor.

In Example 5, the universal force sensor module of example 4 can optionally include a sensor interface structure with a sensor cover contact for contacting the force sensor.

In Example 6, the universal force sensor module of any one of examples 4 or 5 can optionally include a sensor cover contact that produces an interference fit with the force sensor when the upper module cover is joined with the module base.

In Example 7, the universal force sensor module of any one of examples 1 to 6, can optionally include an upper module cover with an instrument contact surface covering a portion of an outer surface of the upper module cover.

In Example 8, the universal force sensor module of example 7 can optionally include a cover flex section with a thin walled section corresponding to the instrument contact surface.

In Example 9, the universal force sensor module of any one of examples 7 and 8 can optionally include an instrument contact surface with a narrower medial/lateral dimension in comparison to a anterior/posterior dimension.

Example 10 can include a surgical system comprising a universal force sensor module and a trial prosthesis. In this example, the universal force sensor module can include an upper module cover, a module base, a force sensor, and a circuit board. In this example, the module base can include a sensor support structure and be configured to receive a cover perimeter section of the upper module cover. The force sensor can be disposed within the module base and be supported by the sensor support structure. The circuit board electrically can be coupled to the force sensor and a battery, and the circuit board and the battery can be disposed within the module base. The trial prosthesis can include a sensor module cavity adapted to securely retain the universal force sensor module.

In Example 11, the surgical system of example 10 can optionally include a set of trial prosthesis each including a sensor module cavity adapted to securely retain the universal force sensor module.

In Example 11, the surgical system of any one of examples 10 or 11 can optionally include a handling instrument adapted to insert the universal force sensor module into the sensor module cavity in the trial prosthesis.

In Example 13, the surgical system of example 12 can optionally include a handling instrument with a plurality of engagement tabs adapted to engage handling scallops in the module base of the universal force sensor module, and a stabilizer adapted to engage at least a portion of the upper module cover when the plurality of engagement tabs are engaged with the handling scallops.

In Example 14, the surgical system of any one of examples 12 or 13 can optionally include a handling instrument with a locking mechanism to positively capture the universal force sensor module.

In Example 15, the surgical system of example 14 can optionally include a locking mechanism with a ratchet-type mechanism to provide various degrees of pressure on the universal force sensor module.

In Example 16, the surgical system of any one of examples 10 to 15 can optionally include an articular insert adapted to fit over the universal force sensor module within the sensor module cavity to provide a substantially uniform articular surface over a portion of the trial prosthesis.

In Example 17, the surgical system of any one of examples 10 to 16 can optionally include a computing device. In this example, the computing device can include one or more processors for processing data and generating a graphical user-interface, a communication circuit for wirelessly communicating with one or more universal force sensors including the universal force sensor module, and a display device for displaying the graphical user-interface.

In Example 18, the surgical system of example 17 can optionally include a graphical user-interface with numeric and graphical information related to the universal force sensor module.

In Example 19, the surgical system of any one of examples 17 or 18 can optionally include a communication circuit adapted to communicate with the one or more universal force sensors over a Bluetooth communication protocol.

Example 20 can include a surgical instrument for determining forces within a joint. The surgical instrument of this example can include an instrument housing and a universal force sensor module disposed within one of the one or more force sensor module cavities. The instrument housing can include one or more force sensor module cavities, and the instrument housing can be configured to position the one or more force sensor module cavities within the joint. In this example, the universal force sensor module can include an upper module cover, a module base, a force sensor and a circuit board. The module base can include a sensor support structure and be configured to receive a cover perimeter section of the upper module cover. The force sensor can be disposed within the module base and be supported by the sensor support structure. The circuit board can be electrically coupled to the force sensor and a battery, the circuit board and battery disposed within the module base.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1A is a three dimensional exploded illustration of a universal force sensor module, according to an example embodiment.

FIG. 1B is a three dimensional perspective view illustration of a universal force sensor module, according to an example embodiment.

FIG. 1C is a cross-section illustration of a universal force sensor module, according to an example embodiment.

FIG. 1D is a three-dimensional perspective view cross-section illustration of a universal force sensor module, according to an example embodiment.

FIG. 1E is a three-dimensional perspective view illustration of a module base of a universal force sensor module, according to an example embodiment.

FIGS. 2A-2B are illustrations of a handling instrument for handling a universal force sensor module, according to an example embodiment.

FIGS. 3A-3C are illustrations of a handling instrument for handling a universal force sensor module, according to an example embodiment.

FIGS. 4A-4C are three-dimensional perspective view illustrations of a provisional tibial implant assembly including universal force sensor modules, according to example embodiments.

FIGS. 5A-5B are three-dimensional perspective view illustrations of a provisional hip implant assembly including a universal force sensor module, according to an example embodiment.

FIG. 6A-6B are three-dimensional perspective view illustrations of a surgical instrument including universal force sensor modules, according to example embodiments.

FIG. 7 is an illustration of a graphical user-interface for monitoring one or more universal force sensors, according to an example embodiment.

FIG. 8 is a flowchart illustrating a method of use for using one or more universal force sensor modules in a surgical procedure, according to an example embodiment.

FIG. 9 is a diagram illustrating a computing device for hosting a graphical user-interface to monitor universal force sensor modules, according to an example embodiment.

### DETAILED DESCRIPTION

Detailed structure, configurations, and methods of use for a universal force sensor module, associated instruments, and provisional components are generally disclosed herein. In one example, a universal force sensor module includes a force sensor, a circuit board, one or more batteries, and a specially designed housing. In another example, provisional components for using universal force sensor modules within knee, hip, or other joint reconstruction surgeries are discussed. For example, during total knee arthroplasty (TKA), a surgeon can use one of the provisional tibial implant assemblies in conjunction with universal force sensors discussed herein to balance soft tissues within a knee before making final resections of the femur and/or tibia. Similarly, universal force sensor modules can be used within an acetabular cup trial component discussed herein verify proper femoral head length or other parameters of a total hip arthroplasty (THA) procedure.

### Definitions:

The following definitions are provided solely to assist the reader in understanding the following detailed description of various examples illustrated by the included figures.

Provisional: A provisional component or assembly is a component or assembly that is similar in shape and fit to the actual implant (prosthesis) that will be used to complete the surgical procedure.

Trial component: A provisional type component or assembly typically provided as part of a surgical kit or system and including multiple different sizes or thickness to assist in determining size of a prosthetic implant to be used as part of a joint reconstruction.

### Universal Force Sensor Module (Assembly):

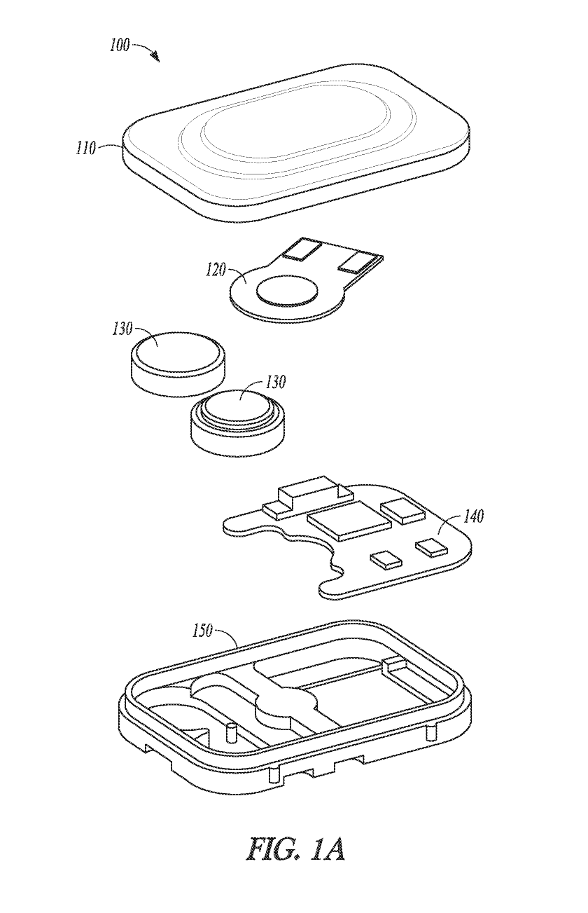

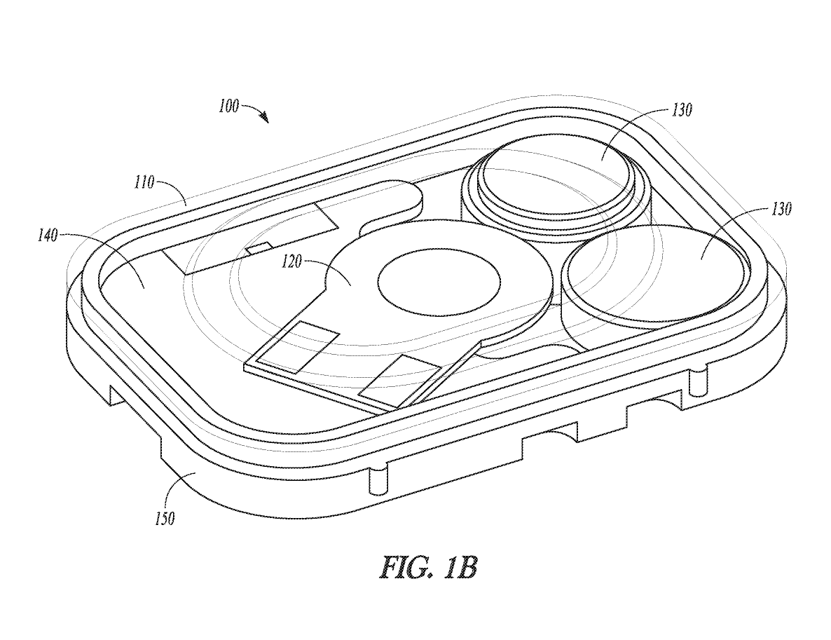

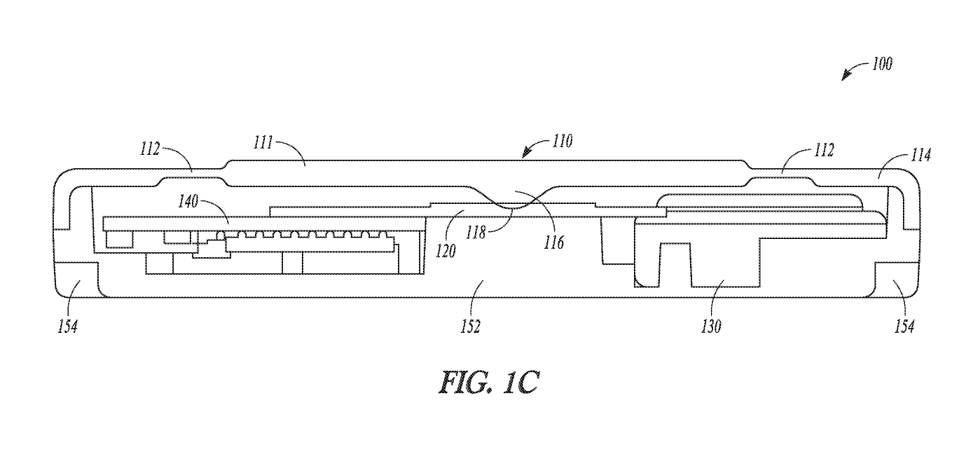

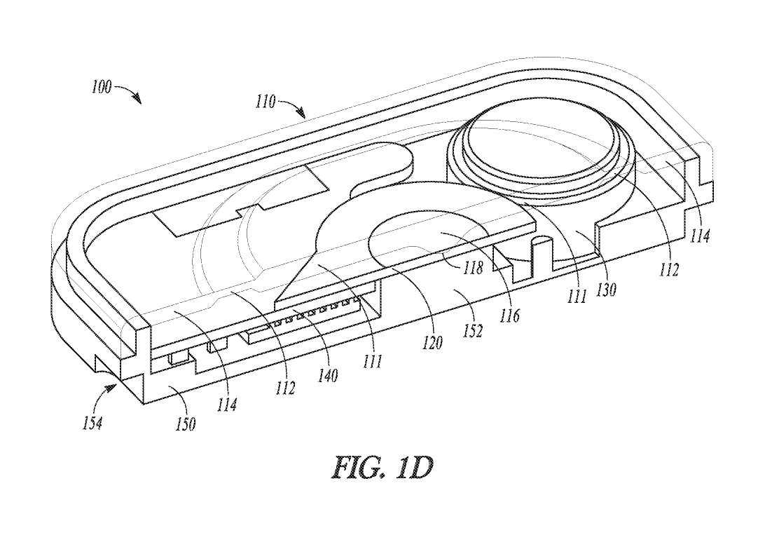

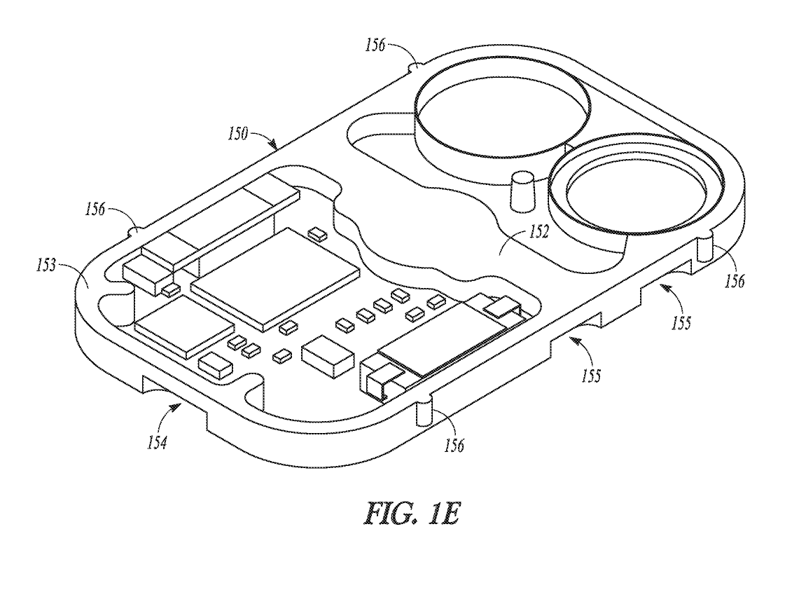

FIGS. 1A-1B are a three dimensional perspective view illustrations of a universal force sensor module 100, according to an example embodiment. In an example, the universal force sensor module includes a module cover 110, a force sensor 120, a battery 130, a circuit board 140, and a module base 150. The module cover 110 (or upper housing) is designed to provide an interface between the force sensor 120 and an articular surface of a provisional or implant component. In some examples, the module cover 110 is designed to interface with natural anatomical structures, such as bone, cartilage, or other hard and soft tissues in the surgical field. The circuit board 130 is electrically coupled to the force sensor 120 and includes processing circuitry as well as communication circuitry. In certain examples, the communication circuitry provides wireless communication capabilities through protocols such as Bluetooth low-energy, Wifi, or near-field communication (NFC), among others. In some examples, the universal force sensor module includes a physical communication and/or battery charging connector, such as micro-USB connector.

FIG. 1C is a cross-section illustration of a universal force sensor module 100, according to an example embodiment. FIG. 1D is a three-dimensional perspective view cross-section illustration of a universal force sensor module 100, according to an example embodiment. The cross-section illustrations depict an example design for the module cover 110 and module base 150 in greater detail. In this example, the module cover 110 includes a cover instrument contact surface 111, a cover flex section 112, a cover perimeter section 114, a sensor interface structure 116, and a sensor cover contact 118. In this example, the universal force sensor module 100 also includes a module base (lower housing) 150 that includes a base cross-beam (sensor support structure) 152 and handling scallops 154.

In the example illustrated in FIG. 1C, the module cover (upper housing) 110 has an instrument contact surface 111 that is raised in comparison to the surrounding module cover 110 to engage an instrument, provisional, or trial component surface. In some examples, the instrument contact surface can be coated with a protective material or include a bonded additional surface to prevent excessive wear from contacting articular surfaces. In the illustrated example, the module cover 110 is a single homogeneous molded component. The material for the module cover 110 is selected to maintain consist flex properties after sterilization. In this example, the module cover 110 includes a cover flex section 112, which is a reduced cross-section area surrounding the instrument contact surface 111. The cover flex section 112 is designed to allow micron-level deformation in the module cover 110, specifically the instrument contact surface 111, in response to pressure exerted on the universal force sensor module 100. In other examples, the cover flex section 112 can cover a wider area or include a narrower cross-section to allow for greater flex in the module cover 110 with respect to the force sensor 120. In an example, the cover flex section 112 can extend over a large percentage of the A/P (anterior/posterior) dimension of the module cover 110 and optionally have a narrow M/L (medial/lateral) dimension. A specific example can include a module cover with a cover flex section covering 80% of the A/P dimension and 20% of the M/L dimension. Creating cover flex sections with different dimensions allows for the sensor module to be adapted to different applications that may require different force sensitive areas. Additional example universal force sensor modules can vary overall dimensions of the entire device in the A/P versus M/L dimensions to conform to the necessary application. Further example universal force sensor modules can include a module cover, such as module cover 110, with a cover flex section or thin membrane covering the majority or entirety of the surface and including an instrument contact surface.

Further illustrated in FIG. 1C are the sensor interface structure 116 and the sensor cover contact 118 portions of the example module cover 110. In this example, the sensor interface structure 116 and the sensor cover contact 118 are designed to concentrate the pressure exerted on the instrument contact surface 111 onto the force sensor 120. In this example, the sensor interface structure 116 is substantially conical with the sensor cover contact 118 rounding out the conical sensor interface structure 116. In other examples, the sensor interface structure 116 and the sensor cover contact 118 can be designed differently to engage the force sensor 120 in a desired manner. For example, the sensor interface structure 116 could be cylindrical with the sensor cover contact 118 forming the end of the cylinder, which would provide a greater contact area on the force sensor 120.

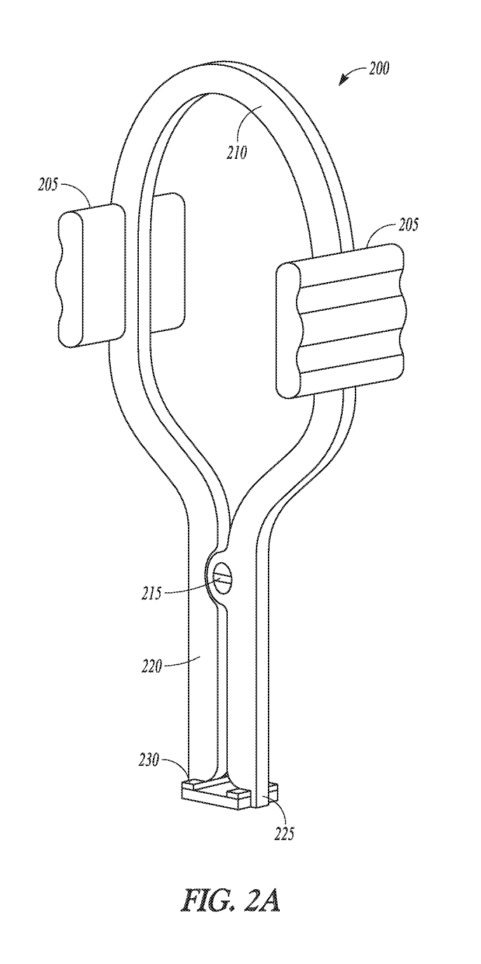

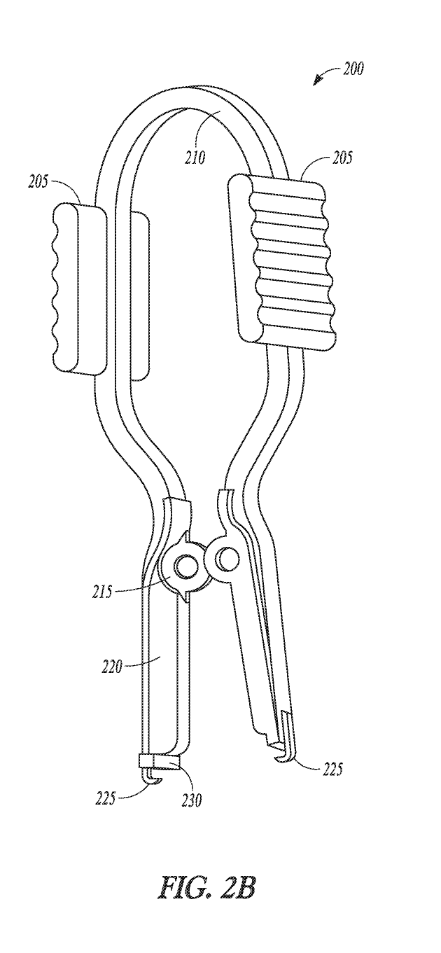

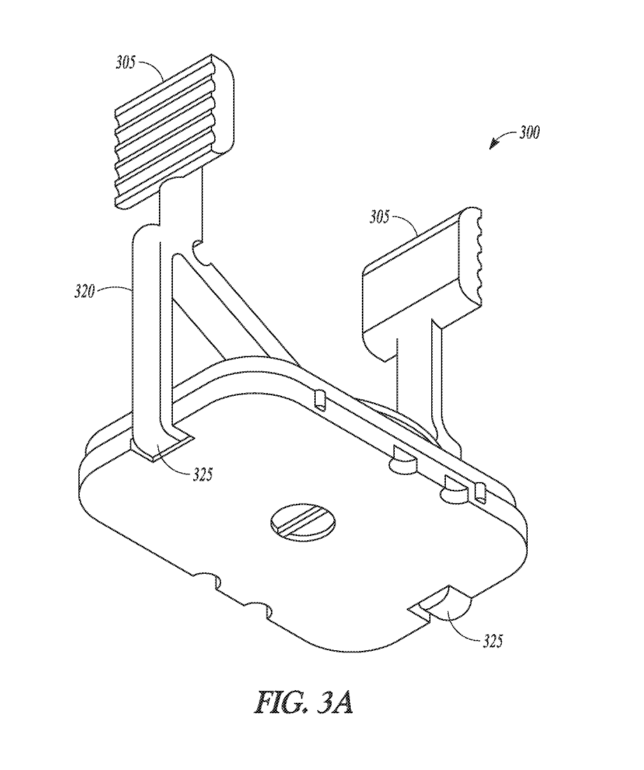

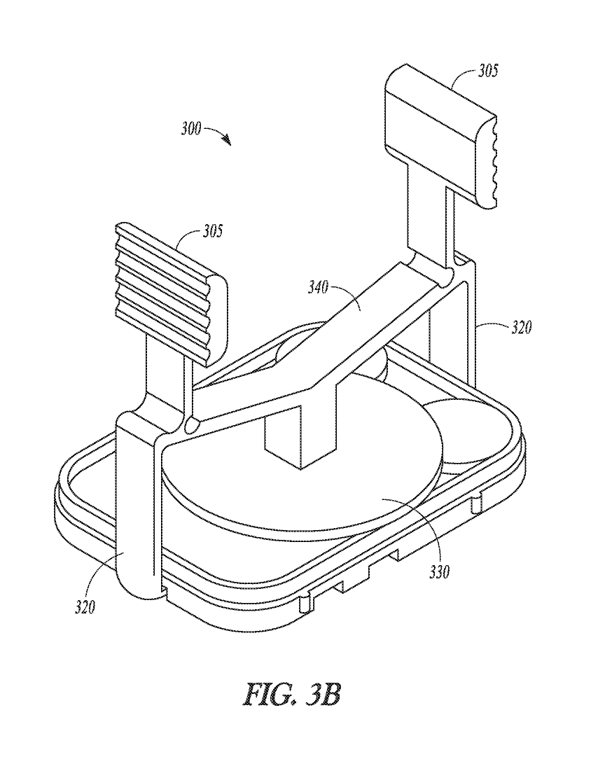

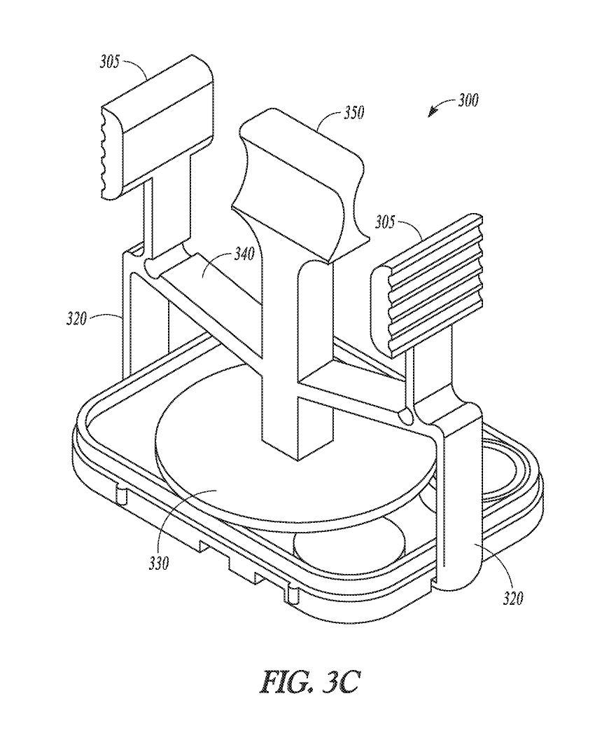

In an example, the module base 150 includes a cross-beam (sensor support structure) 152 to provide a solid stable structure under the force sensor 120. The module base 150 also includes handling scallops 154 on at least two ends of the module base 150 to ease handling of the relatively small device with handling instruments discussed below in reference to FIGS. 2A-3C.

FIG. 1E is a three-dimensional perspective view illustration of a module base 150 of a universal force sensor module 100, according to an example embodiment. In this example, the module base 150 includes a sensor support structure 152, a base perimeter 153, handling scallops 154, instrument scallops 155, and instrument projections 156. The example module base 150 features enable improved handling, control placement, and enhance rigidity of the universal force sensor module 100. The sensor support structure 152 bridges the center of the module base 150 and supports the force sensor 120 to enhance reliability of the force measurements. The instrument scallops 155 provide alignment features to mate with insertion cavities in provisional components, trial components, or surgical instruments using universal force sensor modules. Similarly, the instrument projections 156 can mate with insertion cavity walls or corresponding scallops.

### Universal Force Sensor Module Handling Instruments:

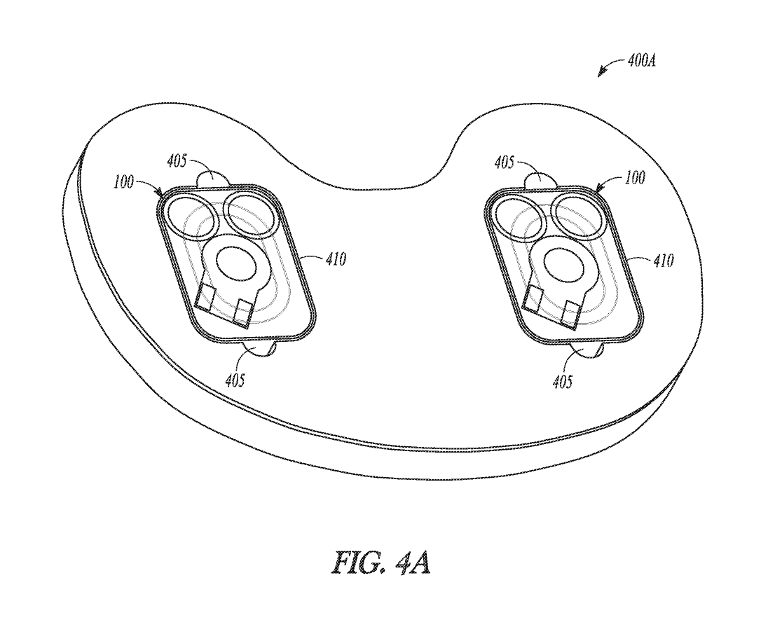

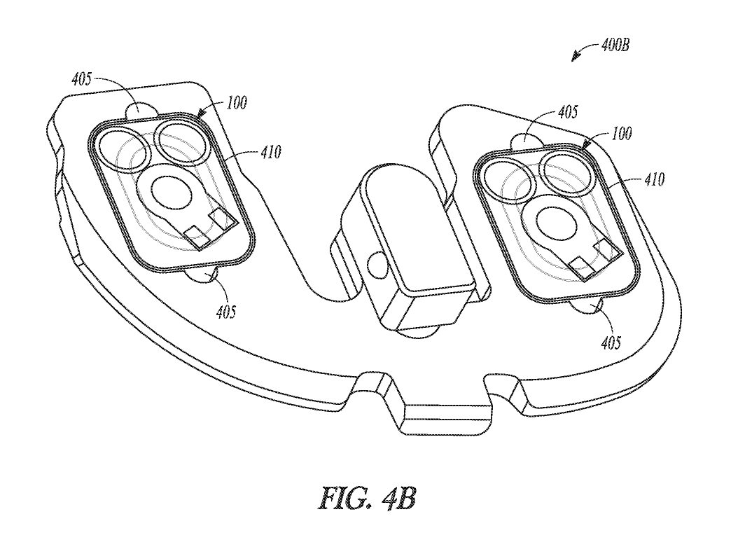

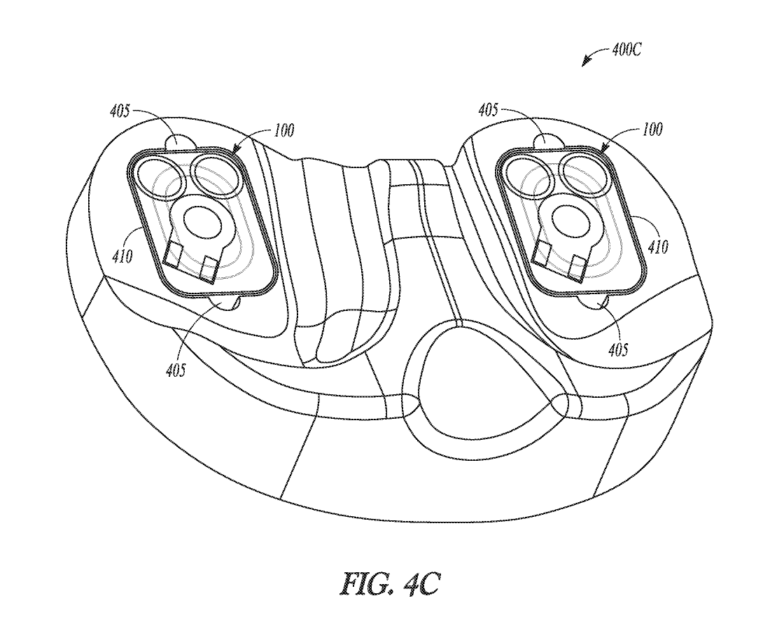

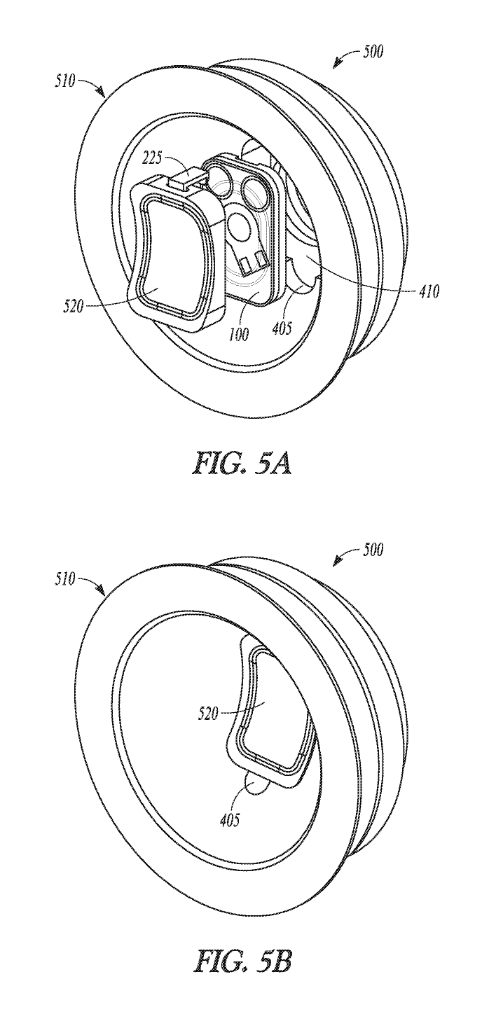

FIGS. 2A-2B are illustrations of a handling instrument 200 for handling a universal force sensor module 100, according to an example embodiment. In an example, handling forceps 200 include manipulation grips 205, a connecting arch 210, a locking member 215, extension legs 220, engagement tabs 225, and stabilizers 230. The handling forceps 200, also referenced as insertion or extraction forceps, are designed to ease handling of the sensor modules prior to or during surgical procedures. For example, the handling forceps 200 are used to insert or extract sensor modules from trial prosthetics, such as those illustrated in FIGS. 4A-5B. In this example, the manipulation grips 205 are used to manipulate the engagement tabs 225 into position on either side of a sensor module. The engagement tabs 225 are configured to positively engage and optionally lock into handling scallops, such as handling scallops 154, in the sides of the sensor module. In this example, the stabilizers 230 engage a portion of the module cover 110 to assist with stabilizing the sensor module during insertion into a cavity within a prosthetic or instrument (see FIG. 4A illustrating a module cavity 410).

In an example, the handling forceps 200 include a locking member 215 designed to engage when a sensor module is secured within the engagement tabs 225. In some examples, the locking member 215 is a ratchet-type locking member that can produce progressively higher amounts of force on the engagement tabs 225 as the manipulation grips 205 are squeezed. A ratchet-type locking member includes multiple lock engagement points or ridges.

FIGS. 3A-3C are illustrations of a handling instrument 300 for handling a universal force sensor module 100, according to an example embodiment. In this example, the handling instrument 300 includes manipulation grips 305, extension legs 320, engagement tabs 325, a stabilizer 330, a connecting beam 340, and optionally a pull handle 350. The handling instrument 300 functions in a generally similar manner to handling forceps 200, in that engagement tabs 325 are configured to securely engage with handling scallops, such as handling scallops 154, in the sensor module body (such as module base 150). In this example, handling instrument 300 includes a large stabilizer 330 that may assist in insertion of the sensor module into tight fitting cavities, such as cavities design to have an interference fit with at least portions of the sensor module.

Prosthesis and Instruments for Use with Universal Force Sensor Module(S):

FIGS. 4A-4C are three-dimensional perspective view illustrations of a provisional tibial implant assembly including universal force sensor modules, according to example embodiments. In the examples illustrated in FIGS. 4A-4C, the provisional tibial inserts 400A, 400B, 400C include, among other features, insertion/extraction scallops 405 (insertion scallops), module cavities 410 and universal force sensor modules 100. The insertion scallops 405 are formed to enable use of one of the handling instruments discussed above for insertion and optionally extraction of universal force sensor modules. The module cavities 410 are formed to positively position and secure the universal force sensor module within the provisional tibial insert. The example provisional tibial inserts 400A, 400B, 400C can optionally include articular inserts to cover the universal force sensor modules if the particular procedure or application indicates additional protection may be needed for the force sensors.

FIGS. 5A-5B are three-dimensional perspective view illustrations of a provisional hip implant assembly 500 including a universal force sensor module 100, according to an example embodiment. In this example, the provisional hip implant assembly 500 includes an acetabular cup provisional 510, an articular insert 520, and a universal force sensor module 100. As noted above, articular inserts, such as articular insert 520, can be used to protect a universal force sensor 100. However, articular inserts can also be used to provide a desired shape for an articular surface for a particular application, such as the hip implant assembly 500 that maintains a spherical or similar three-dimensional surface in the area where force or pressure detection is desired. Accordingly, the articular insert 520 is formed to match the surrounding surface of the acetabular cup provisional 510 when properly positioned over the universal force sensor module 100. The articular inserts can also include engagement tabs, similar to engagement tabs 225, to positively engage with the module base 150 of the universal force sensor 100 (as illustrated in FIG. 5A).

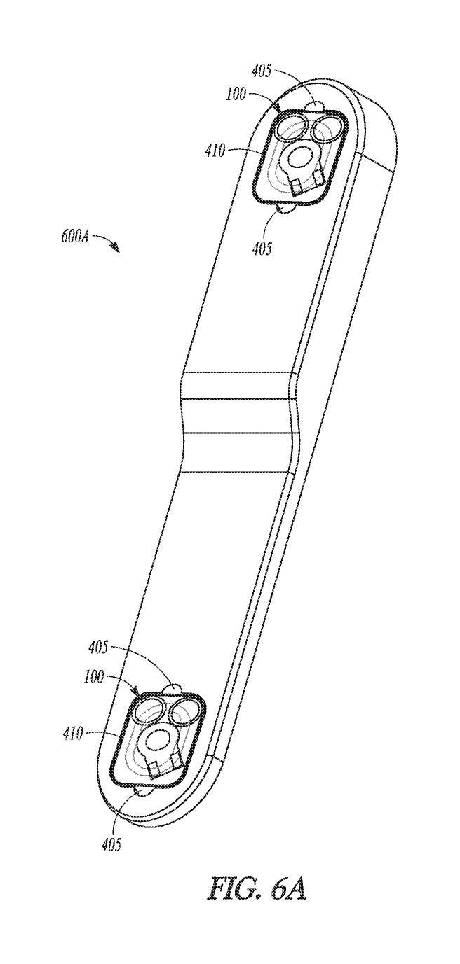

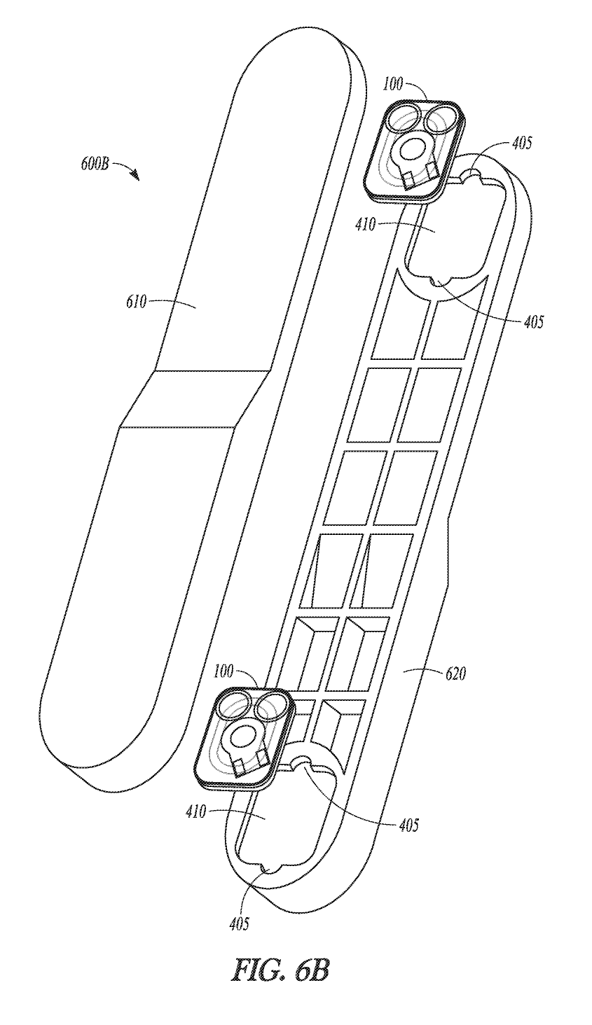

FIGS. 6A-6B are three-dimensional perspective view illustrations of a surgical instrument including universal force sensor modules, according to example embodiments. In the examples illustrated in FIGS. 6A-6B, gauge instruments 600A and 600B include, among other features, insertion/extraction scallops 405 (insertion scallops), module cavities 410 and universal force sensor modules 100. The gauge instrument 600A is an example shim or sizing instrument that includes two different thicknesses each with a universal force sensor module positioned to measure forces within a joint or similar anatomical feature. Gauge instrument 600A illustrates an instrument with exposed universal force sensor modules, while instrument 600B is a clamshell design with the universal force sensor sandwiched between an upper housing 610 and a lower housing 620. The illustrated instruments represent two examples of a wide range of surgical instruments that could be fitted with one or more universal force sensor modules.

### Example Graphical User-Interface

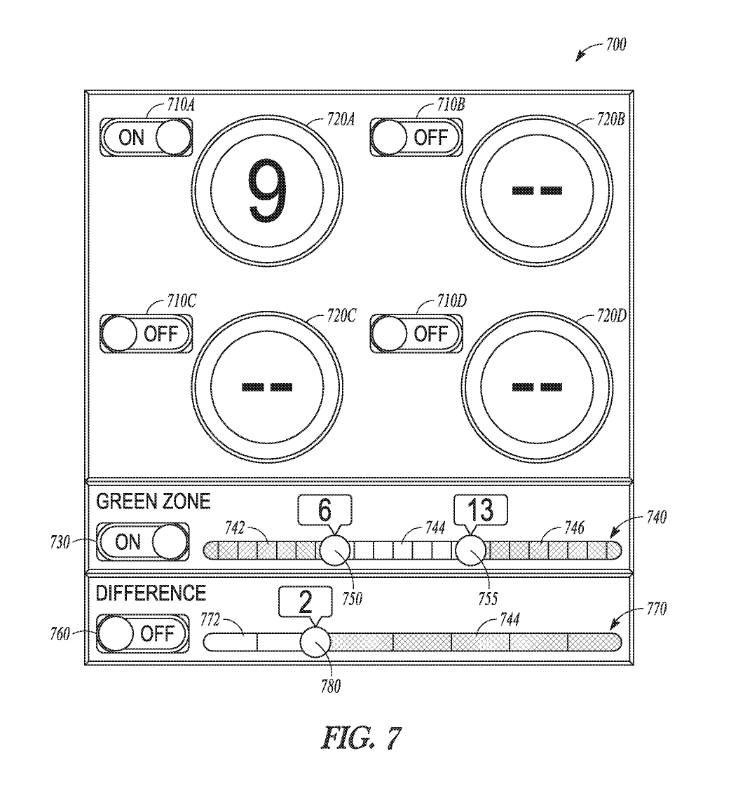

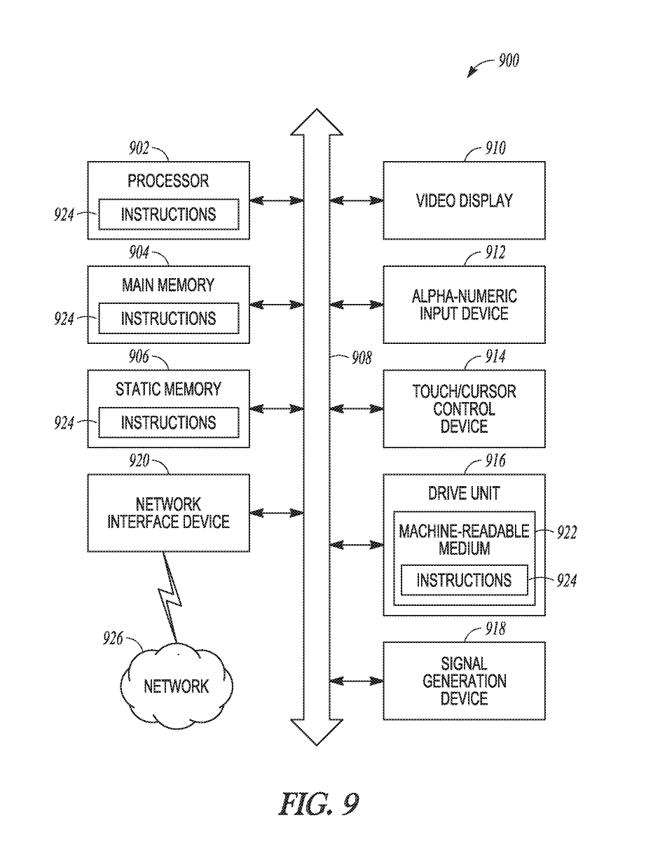

FIG. 7 is an illustration of a graphical user-interface 700 for monitoring one or more universal force sensors, according to an example embodiment. In an example, the graphical user-interface (GUI) 700 includes multiple sensor switches (710A, 710B, 710C, 710D, collectively referred to as sensor switches 710), multiple sensor readouts (720A, 720B, 720C, 720D, collectively referred to as sensor readouts 720), a green zone switch 730, a green zone range controls 740, a green zone low range 742, a green zone acceptable range 744, a green zone high range 746, a green zone high range 746, a green zone low control 750, a green zone high control 755, a difference switch 760, a difference range 770, a difference acceptable range 772, a difference high range 774, and a difference control 780. Optionally, the GUI 700 can include more or fewer sensor switches 710 and sensor readouts 720. In an example, sensor switches 710 and sensor readouts 720 are added to the GUI 700 dynamically in response to connecting each universal force sensor module 100 to a host computing device (such as the device illustrated in FIG. 9). In another example, force data from one or more universal force sensor modules can be displayed on a dedicated wireless display device. The dedicated wireless display device can be a simple numeric display using LEDs, e-ink, or other suitable display technologies. In an example, the dedicated wireless display device can be a small, single-use sterile disposable device packaged with one or more universal force sensor modules, or available as an optional accessory.

The sensor switches 710 activate the sensor readouts 720 for the selected sensor. In some examples, the universal force sensor modules can be named as they are connected to the host computing device. In these examples, the GUI 700 can report the names of the individual sensor modules allowing the user to better understand which sensor modules are being controlled or readout.

The Green Zone area of the GUI 700 controls the color of the sensor readouts 720 based on the force or pressure reading received from the corresponding universal force sensor module. When activated via green zone switch 730, the Green Zone controls enable a user to set upper and lower bounds for sensor readings via the green zone range controls 740. The lower bound is controlled via the green zone low control 750 and displayed within a combination of green zone low range 742 and green zone acceptable range 744. Similarly, in this example, the upper bound of the green zone is controlled through manipulation of the green zone high control 755 and displayed through a combination of the green zone high range 746 and the green zone acceptable range 744.

The Difference area of the GUI 700 provides a different control over the behavior of the sensor readouts 720. The Difference area enables a visual indication or other form of alert (pop-up, audible, etc . . . ) based on a detected difference in force readings from enabled universal force sensor modules. When activated via difference switch 760, the difference range controls 770 allow a user to select the desired limits on difference between sensor module readings. In this example, the difference range controls 770 include a difference acceptable range 772, a difference high range 774, and a difference control 780. The difference control 780 in this example is a slider control that allows for setting a maximum acceptable difference between sensor readings. In another example, not illustrated, the difference controls 770 can include controls to select specific universal force sensor modules to include in the difference calculations. Additionally, examples can optionally also include multiple sets of difference controls 770 to enable different difference settings for different pairs of sensors, for example.

### Example Method of Use

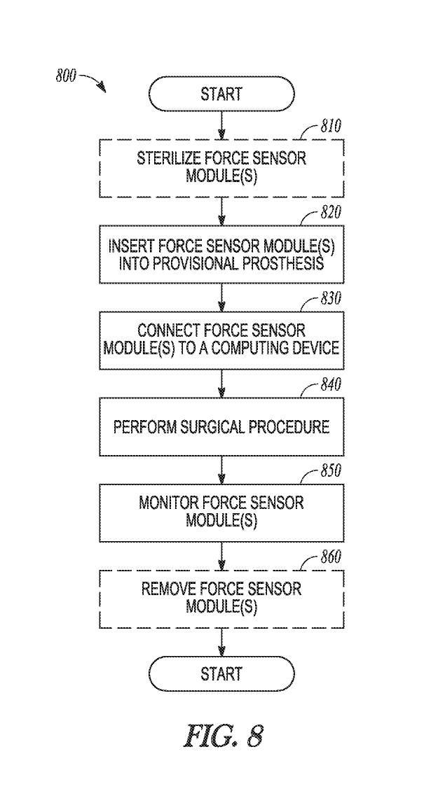

FIG. 8 is a flowchart illustrating a method of use 800 for using one or more universal force sensor modules in a surgical procedure, according to an example embodiment. In an example, the method 800 includes operations such as sterilizing a force sensor module at 810, inserting the force sensor module into a provisional prosthesis at 820, connecting the force sensor module to a computing device hosting a graphical user-interface (GUI) at 830, performing a surgical procedure at 840, monitoring the force sensor module on GUI at 850, and optionally removing the force sensor module from the provisional prosthesis at 860.

In this example, the method 800 optionally begins at 810 with a member of the surgical team sterilizing the force sensor module. As the force sensor modules will typically be pre-sterilized single use devices, operation 810 may be unnecessary in most example procedures. However, as sterilization methods and materials for use in manufacturing a force sensor module, such as the universal force sensor module 100, advance multiple use devices are envisioned. At 820, the method 800 continues with a member of the surgical team using a handling instrument, such as handling forceps 200, to insert the force sensor module into a provisional prosthesis, such as the provisional tibial insert 400A. At 830, the method 800 continues with the inserted force sensor module being communicatively connected to a computing device hosting a GUI for monitoring data sent by the force sensor module. In an example, connecting the force sensor module can involve a Bluetooth or near-field communication (NFC) pairing operation. In another example, the force sensor module can be configured to automatically detect and connect to a host computing device over Bluetooth, NFC, Wifi, or some similar wireless networking protocol.

Once connected, the method 800 continues at 840 with the surgical team performing the intended surgical procedure, or at least the portion of the procedure involving the provisional prosthesis with force sensor module inserted. During the procedure, the method 800 continues at 850 with the surgical team monitoring data transmitted from the force sensor module to the host computing device via the GUI. As described above, the GUI 700 provides both numeric and visual feedback to the surgical team based on forces sensed by the one or more force sensors being monitored. The numeric and visual feedback is used by the surgical team to modify aspects of the procedure to obtain a more favorable outcome for the patient. Finally, method 800 can conclude at 860 with a member of the surgical team removing the force sensor from the provisional prosthesis.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive.

For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. A universal force sensor module comprising:
an upper module cover including a cover flex section;
a module base including a sensor support structure, the module base configured to receive a cover perimeter section of the upper module cover;
a force sensor disposed within the module base and supported by the sensor support structure; and
a circuit board electrically coupled to the force sensor and a battery, the circuit board and battery disposed within the module base.

2. The universal force sensor module of claim 1, wherein the cover flex section includes a reduced thickness portion surrounding a cover instrument contact surface.

3. The universal force sensor module of claim 2, wherein the reduced thickness portion of the cover flex section allows for micron-level defections in the cover instrument contact surface in a direction substantially perpendicular to a plane formed by the outer surface of the upper module cover.

4. The universal force sensor module of claim 2, wherein the upper module cover further includes a sensor interface structure disposed on the inside surface and configured to contact the force sensor.

5. The universal force sensor module of claim 4, wherein the sensor interface structure includes a sensor cover contact for contacting the force sensor.

6. The universal force sensor module of claim 5, wherein the sensor cover contact produces an interference fit with the force sensor when the upper module cover is joined with the module base.

7. The universal force sensor module of claim 1, wherein the upper module cover includes an instrument contact surface covering a portion of an outer surface of the upper module cover.

8. The universal force sensor module of claim 7, wherein the cover flex section is a thin walled section corresponding to the instrument contact surface.

9. The universal force sensor module of claim 7, wherein the instrument contact surface has a narrower medial/lateral dimension in comparison to a anterior/posterior dimension.

10. A surgical system comprising:
a universal force sensor module, the module comprising:
an upper module cover;
a module base including a sensor support structure and configured to receive a cover perimeter section of the upper module cover;
a force sensor disposed within the module base and supported by the sensor support structure; and
a circuit board electrically coupled to the force sensor and a battery, the circuit board and battery disposed within the module base; and
a trial prosthesis including a sensor module cavity adapted to securely retain the universal force sensor module.

11. The surgical system of claim 10, further comprising a set of trial prosthesis each including a sensor module cavity adapted to securely retain the universal force sensor module.

12. The surgical system of claim 10, further comprising a handling instrument adapted to insert the universal force sensor module into the sensor module cavity in the trial prosthesis.

13. The surgical system of claim 12, wherein the handling instrument includes:
a plurality of engagement tabs adapted to engage handling scallops in the module base of the universal force sensor module; and
a stabilizer adapted to engage at least a portion of the upper module cover when the plurality of engagement tabs are engaged with the handling scallops.

14. The surgical system of claim 12, wherein the handling instrument includes a locking mechanism to positively capture the universal force sensor module.

15. The surgical system of claim 14, wherein the locking mechanism includes a ratchet-type mechanism to provide various degrees of pressure on the universal force sensor module.

16. The surgical system of claim 10, further comprising an articular insert adapted to fit over the universal force sensor module within the sensor module cavity to provide a substantially uniform articular surface over a portion of the trial prosthesis.

17. The surgical system of claim 10, further comprising a computing device, the computing device including:
one or more processors for processing data and generating a graphical user-interface;
a communication circuit for wirelessly communicating with one or more universal force sensors including the universal force sensor module; and
a display device for displaying the graphical user-interface.

18. The surgical system of claim 17, wherein the graphical user-interface includes numeric and graphical information related to the universal force sensor module.

19. The surgical system of claim 17, wherein the communication circuit is adapted to communicate with the one or more universal force sensors over a Bluetooth communication protocol.

20. A surgical instrument for determining forces within a joint, the surgical instrument comprising:
an instrument housing including one or more force sensor module cavities, the instrument housing configured to position the one or more force sensor module cavities within the joint; and
a universal force sensor module disposed within one of the one or more force sensor module cavities, the universal force sensor module including:
an upper module cover;
a module base including a sensor support structure and configured to receive a cover perimeter section of the upper module cover;
a force sensor disposed within the module base and supported by the sensor support structure; and
a circuit board electrically coupled to the force sensor and a battery, the circuit board and battery disposed within the module base.
